(19) United States
(12) Patent Application Publication
(10) Pub. No.: US 2015/0059449 A1
(43) Pub. Date: Mar. 5, 2015

(54) New Lithographic Method

(71) Applicant: Technische Universiteit Delft, Delft (NL)

(72) Inventors: Qiang Xu, Delft (NL); Gregory Schneider, Delft (NL); Henny Zandbergen, Delft (NL); Mengyue Wu, Delft (NL); Bo Song, Delft (NL); Cornelis Dekker, Delft (NL)

(21) Appl. No.: 14/478,620

(22) Filed: Sep. 5, 2014

Related U.S. Application Data
(63) Continuation of application No. PCT/NL13/50136, filed on Mar. 4, 2013.

(30) Foreign Application Priority Data
Mar. 5, 2012 (NL) 2008412

(51) Int. Cl. G01N 33/487; B81C 1/00

(52) U.S. Cl. CPC G01N 33/48721; B81C 1/00531; B81C 2203/00
USPC 73/61.43; 216/94; 428/141

(57) ABSTRACT

A method for removing a high definition nanostructure in a partly free-standing layer, the layer, a sensor comprising said layer, a use of said sensor, and a method of detecting a species, and optional further characteristics thereof, using said sensor. The sensor and method are suited for detecting single ions, molecules, low concentrations thereof, and identifying sequences of base pairs, e.g., in a DNA-strand.

### CROSS-REFERENCE TO RELATED APPLICATIONS

This application is a continuation application of Patent Cooperation Treaty Application No. PCT/NL2013/050136, filed Mar. 4, 2013, entitled “Method for Removing a High Definition Nanostructure, a Partly Freestanding Layer, a Sensor Comprising Said Layer and a Method Using Said Sensor”, which claims priority to Netherlands Patent Application Serial No. 2008412, filed Mar. 5, 2012, entitled “New Lithographic Method”, and the specifications and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

### Field of the Invention (Technical Field)

The present invention relates to a method for removing a high definition nanostructure in a partly free-standing layer, the layer, a sensor comprising said layer, a use of said sensor, and a method of detecting a species, and optional further characteristics thereof, using said sensor. The sensor and method are suited for detecting single ions, molecules, low concentrations thereof, and identifying sequences of base pairs, e.g., in a DNA-strand.

Graphene has attracted a lot of research interest because of its promising electronic applications related to its superior electron mobility, mechanical strength and thermal conductivity. It may have wide range of applications, for instance, field-effect transistors, photonic or optoelectronic device, sequencing DNA through nano-holes in graphene etc. Most of these applications demand modification of a graphene sheet into specific nano-patterns.

In a published paper, inventors used a transmission microscope operating in high resolution mode to sculpt graphene, showing a possibility of sculpting for multi-layer graphene. It was also found that for monolayer graphene, nanometer precision sculpting could not be obtained using the same method due to a width of an electron beam and unpredicted e-beam damage during an accompanying imaging process.

Various documents recite production of nanostructures. For instance, B. Song et al, in ‘Atomic-Scale Electron-Beam Sculpting of Near-Defect-Free Graphene Nanostructures’, 2011, 11 (6), pp. 2247-2250, recites fabrication of a range of graphene nanostructures into multilayer graphene, also known as graphite. In the method no contamination occurs upon electron beam exposure, and the graphitic material remains crystalline up to edges of a nanostructure formed (i.e., the graphite does not get amorphous). However, the nanostructure formed cannot be programmed in advance (for example using a computer script). A further disadvantage is that upon electron beam exposure that nanostructure will change in shape, fold, etc. It is noted that, although the publication recites graphite, it recites a multilayer graphene which is physically and chemically very different from the unique mono- or bilayer graphene. In this respect it is e.g. noted that if the method in the paper is applied to monolayer graphene (i.e., that lithography is carried out in a bright field mode of an electron microscope), it has been found that the material is extremely sensitive upon electron beam exposure, making it virtually impossible to fabricate a predetermined structure in the material; e.g., even a simple hole (e.g. 5 nm in diameter) could not be made (see FIG. 1, showing that if one hole is intended in the bright field mode, always several holes appears, making the above technique for graphene nanostructure design useless.

Further, e.g., WO2011/046706 recites use of a nanopore in graphene for DNA analysis. A transmission electron microscope in the bright field mode is used.

The graphene undergoes the problems raised in the above publication, namely contamination, amorphization and lack of control. Also, use is made of synthetic graphene which is generally multilayer graphene. The method does not provide perfect control and reproducibility. It is not possible to produce controlled and perfectly crystalline nanopores in single layer graphene.

In CN101872120 a method for preparing patterned graphene is recited. In the method, a photoresist is patterned on a device substrate by a microelectronic process such as UV lithography, and windows are formed at positions needing graphene; by a graphene transfer method, large-area graphene is transferred onto the patterned photoresist; and the photoresist and the graphene thereon are stripped to obtain a patterned graphene. Compared with the prior art, the method has the advantages of accurate positioning, and does not require etching or manufacturing an imprint template so as to have low cost.

However, UV lithography does not allow the fabrication of features smaller than approximately a wavelength of light used (UV, e.g., 200 nm). No single atom resolution is provided. Further, edge structures will be amorphous and contaminated. Third, graphene needs a support, typically a wafer or a resist, constituting another source of contamination.

In Liu et al., ‘Nanosphere Lithography for the Fabrication of Ultranarrow Graphene Nanoribbons and On-Chip Bandgap Tuning of Graphene’, Advanced materials, 2011, 23, 1246, an approach for high throughput, rapid, and low-cost fabrication of ultranarrow graphene nanoribbons (GNRs) using nanosphere lithography (NSL) nanopatterning in combination with low-power O2 plasma etching is presented.

It is noted that nanospheres therein are deposited on graphene acting as a mask. An oxygen plasma is used to etch graphene that is not protected by the nanosphere. By principle a plasma (especially O2 plasma) is reacting (in this case oxidizing) the graphene surface and importantly edges thereof as well, which convert into graphene oxide (an insulator while graphene is conducting). Therefore the chemical nature of graphene (sp2, honeycomb bonded graphene) is changed.

In general it is noted that current techniques to fabricate any given shape in graphene lack required sub-nanometer precision for obtaining atomically sharp and con-trolled regular edges, with an appropriate crystal orientation. They also lack the control over the shape to be made. Even further, the graphene, especially a thin layer thereof, such as a mono-layer, cannot be sculpted using prior art techniques, without destroying at least part of the graphene.

The present invention therefore relates to a method which overcomes one or more of the above disadvantages, without jeopardizing functionality and advantages, as well as products obtainable thereby, and use of said products.

### BRIEF SUMMARY OF THE INVENTION

The present invention relates in a first aspect to a method for removing a high definition nanostructure in a partly free-standing layer, a layer obtainable accordingly, a sensor comprising said layer, use of the sensor and a method of detecting.

Therewith a solution has been found to fabricate a wide-range of nanostructures (e.g., graphene) with structural control at atomic level, without inducing amorphization and without contamination. It was so far impossible to controllably pattern e.g. graphene with atomic resolution.

The present invention relates, e.g., to formation of nano-ribbons or nano-pores with desired sizes and precision, or desired crystallographic orientation, for instance, a nano-ribbon with zigzag edges, requiring to cut along a crystallographic (e.g. [100]) direction. Typically the structure comprises one or more edges, such as an edge along a crystallographic direction of a monolayer. The structure may comprise a geometry, such as circular, hexagonal, triangular, etc. The size of the structure is typically from less than 1 nm, e.g., 1 atom, to a few hundred nm. It is noted that in principle also larger structures, comprising nano- and/or microstructures, can be made using the present method, such as a MEMS.

It is noted that the present method is also applicable using more than one radiation sources, such as 2 or more. Using e.g. software multiple beams can be used to sculpt structures parallel in time. Such is e.g. extremely useful when sculpting repetitive structures, such as a sequence of nano-holes, e.g., in one or two dimensions.

The precision is in the order of 1 atom (e.g., 0.1 nm) or better (0.05 nm), whereas the relative determination of a location is also in the order of 1 atom. That is a radiation source can be focused on such a small area (0.1 nm) relative to a known or predetermined location (x,y). Such requires precise control of heating of the sample, and damping of external factors, such as vibration. Such may also require also forming of an image during sculpting or in between sculpting. It is noted that theoretical calculations show that properties of a graphene device depend very strongly on the exact geometry at nano scale. At present a highly accurate and highly reproducible technique is provided to make a range of such geometries, e.g., for testing theoretical predications. A way to achieve this is to use electrons or other radiation damage to sculpt graphene into desired nano patterns; it is noted that a state of art process does not precisely control radiation damage, contrary to the present invention.

Inventors demonstrate that by using e.g. scanning transmission electron microscopy, surprisingly one can fully control the e-beam induced damage and combine such with a self-repairing effect of graphene at elevated temperature higher than e.g. 500° C. Thereby inventors achieved site and orientation specific nano-scale pattern sculpting of mono-layer graphene with reproducibly for the first time.

By using e.g. a scanning electron probe, graphene could be sculpt into ail kinds of e.g. pre-defined patterns with sub-nanometer resolution (precision) and simultaneously form an image of the sculpted result in same resolution. The present invention therewith provides a full control of (electron) radiation damage of graphene such that the destructive nature of sculpting and in principle non-destructive imaging can be achieved in a same mode (electron beam current, beam energy, etc.), without further a need for e.g. adjustments and alignment. Thus an imaging feed-back controlled sculpting system is provided, that allows automatically fast pattern writing on graphene, and is suitable for large scale graphene device fabrication.

At present it is to the knowledge of the inventors impossible to predefine and then fabricate graphene nanostructures with sub-nanometer accuracy. More importantly it is not possible to program such a structure using a computer script, as is provided by the present invention. The computer script provides e.g. for at least one predefined structure, communication with the radiation source, processing of an optional image formed, feedback to the radiation source, optimization of e.g. sculpting steps to be performed, control, e.g. of heating, focusing, localization of radiation, and quality determination, e.g. in terms of shape, size and location of a structure. Nanometer size graphene nanostructure with sub-nanometer resolutions are for example important in nanoscience and bionanoscience. One particular example is in the field of biomolecule analysis with nanopores and nanogaps. Other examples include narrow sub-nanometer electronics.

Although the present method may appear to be very simple to use, the present invention is not obvious, even for a skilled artisan in the field. For instance, sculpting into monolayer graphene has been made possible only by combining heating of a sample and self-repair thereof and a scanning (electron) probe.

The present invention also relates to a design of a software platform e.g. to allow more parameters to be tuned during sculpting (shapes, different beam sizes, different exposition times per exposed spots). It also relates to sculpting nanostructures on a substrate (using electron or ion beams), It can be scaled-up to (12″) wafer scales. Further a combination of electrical measurement and sculpting is provided. Also atomic resolution sculpting is provided, i.e., one can design defects on graphene atom by atom. Such is regarded totally unique.

A nanostructure typically relates to a structure of intermediate size from molecular to microscopic (micrometer-sized) structures. In describing nanostructures one may differentiate between the numbers of dimensions on the nanoscale, e.g., a nanotextured surfaces, nanotube, nano-particle, etc. Typical dimensions are between 0.1 and 100 nm; its length could be much greater. The present layer, however, typically has dimensions of a few mm width and length up to a few cm, and a thickness in the nanoscale.

It is noted that terms as “upper”, “lower”, etc. are relative terms.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 shows a microscope image;

FIG. 2A shows a schematic layout of a microscope;

FIGS. 2B-2G show microscope images;

FIGS. 3A-3B show microscope images;

FIGS. 4A-4C show microscope images;

FIGS. 5A-5D show microscope images; and

FIGS. 6A-6B represent an example of the method of the invention.

### DETAILED DESCRIPTION OF THE INVENTION

The present invention relates in a first aspect to a method for removing a high definition nanostructure according to claim 1.

The present invention provides a heating means having a shift of less than 0.1 nm/10 sec. Such provides for sculpting with the present accuracy.

The support typically is an electrical insulator, such as SiN. Therewith an electrical current, if a voltage is being applied, will mainly run through the conducting layer.

The present method is extremely clean, e.g., hardly any or no impurities are introduced. Such is essential for the characteristics of the layer. Further, almost no carbon contamination is produced as well.

In an exemplary embodiment the microscope is operated at 20-2500 kV, preferably at 50-1000 kV, more preferably at 100-500 kV, such as at 200-400 kV. If a relatively low voltage is applied, such as with a SEM, a gas may be present to assist sculpting, for instance water vapor may be added. It has been found that a somewhat lower voltage causes less damage.

In an exemplary embodiment the current of the microscope is 0.05-10 nA, e.g. 0.25-2 nA.

In an exemplary embodiment the sample is modified defect free on nanometer scale, in a time frame of less than 500 ms, preferably from 5-250 ms, more preferably from 10-100 ms, such as 20-50 ms.

In an exemplary embodiment the radiation dose is less than 10⁹ “items”/atom, wherein items relates to, e.g., number of electrons, ions, and the like.

In an exemplary embodiment the microscope for forming a nanostructure in a monolayer, comprises a vacuum chamber, and a means for holding the sample to be provided, such as a stage. Such a holder is especially designed by the inventors, in order to obtain desired characteristics of the nanostructures.

In an exemplary embodiment the means for heating is one or more coils, such as a Pt-coil. It has been found that a Pt-coil provides superior reproducibility and reliability.

In an exemplary embodiment the microscope comprises a further source, such as a combination of ions and electrons. There with multiple sources may be provided, each being capable of sculpting. Also a first source may be used for sculpting, and a second for imaging.

In an exemplary embodiment of the present method the radiation source is an electron gun of an electron microscope, preferably a SEM, a HREM, a TEM, a HRTEM, a HRSTEM, and combinations thereof, such as a STEM, HREM and SEM, and STEM and HRSTEM. Although other radiation sources may be applicable, such as a focused ion-beam (FIB), such as using He, or Ga, an electron microscope is preferred in view of higher accuracy. The abbreviations above relate to Scanning Electron Microscope, High Resolution Electron Microscope, Transmission Electron Microscope, High Resolution Transmission Electron Microscope, and High Resolution Scanning Transmission Electron Microscope, respectively. These terms are considered well known in the field, whereas typical features of the invention are not known in the field, at least not in the combinations as claimed.

In an exemplary embodiment of the present method radiation is focused to an area of less than 2 nm, such as less than 1 nm, such as less than 0.1 nm. Effectively atoms can be removed one by one. Some care has to be taken not to damage the layer; therefore the dwell time is preferably limited.

It is preferred that markers are provided on the graphene and/or on the support, in order to improve positioning of the sample. The markers may for instance be a multiple of horizontal and vertical lines, spaced apart, or likewise diagonal lines.

In an exemplary embodiment of the present method an energy used for removing one atom in the layer from 1*10⁻¹⁸ J-1*10⁻¹⁶ J, preferably from 2*10⁻¹⁸ J-5*10⁻¹⁶ J, more preferably from 3*10⁻¹⁸ J-1*10⁻¹⁶ J. It has been found that surprisingly a relatively low energy level is sufficient to remove e.g. atoms, i.e. sculpt a nanostructure. It has also been found experimentally that the present method involves a chance process, in that radiation, e.g., electrons, have a certain chance of “hitting” e.g. an atom, and thereby sculpting said atom. It has been established by inventors that said chance is relatively small, e.g. 10⁻⁹-10⁻⁴, in other words in an example only one out of very many electrons hit an atom. It has also been found experimentally that only a fraction of the energy of a radiation species is transferred to the layer, e.g., to an atom. Said energy fraction is in the order of 10⁻⁶-10⁻³, depending on species used more or less energy of said species is transferred to e.g., an atom. Further, it has been found that the amount of energy transferred does not depend linearly on the energy of a species, e.g., higher energies may provide a lower transfer. Also due to, e.g., temperature fluctuation, e.g., a position of an atom may vary, at least on a nanometer scale, and therefore a focused bundle may be focused (slightly) on a wrong position, e.g. not exactly on an atom, or nucleus thereof. By carrying experiments and/or calculations suitable values for energy, current, dwell-time, eta have been obtained, as, e.g., detailed throughout the description.

In an exemplary embodiment of the present method sculpting per single point is performed during a period of 0.01-1000 mseconds, preferably from 2-500 mseconds, such as from 5-300 mseconds. Examples of times used are 10, 25, 35, 50, 82, 100, 120, and 250 mseconds. The process of sculpting (at a certain point or location) can be interrupted by a time for forming an image, e.g., of a surrounding area. Typically a size of said location is in the order of a few atoms, or 1 atom, such as 1 nm, or less. The image forming time is typically in the order of 1-1000 μseconds, such as 2-500 μseconds, e.g. 5-100 μseconds. It is preferred that image forming takes place in a time small enough to allow the layer to relax. Thereafter sculpting may be continued, e.g., until a desired structure is sculpted. Throughout the present application a time between sculpting period is also referred to as a “dwell time”.

Some examples of settings for an EM are 500 ms/nm at a beam current of 0.15 nA (3 Å resolution), 2 ms/nm at beam current of 5 nA (1 Å resolution), about 2 nm/s at beam current of 0.15 nA current, with 3 Å resolution, and 500 nm/s at a beam-current of 5 nA, with 1 Å resolution.

In an exemplary embodiment of the present method after focusing d) the radiation bundle is moved to a next position on the layer, and wherein optionally steps c) and d) are repeated.

In an exemplary embodiment of the present method the bundle is moved from a first to a further position, which movement is repeated from 1-10*10⁹ times. Also the shape of the nanostructure may. be adapted accordingly. As such a single atom may be removed. In an example a complete structure may be removed, such as a structure wherein a relative large number of atoms is removed, e.g., 10¹⁰ atoms. Typical structures sculpted may have dimensions in the order of nm by nm to 500 μm by 500 μm.

In an exemplary embodiment of the present method further an image is formed of the layer, such as by detecting forward or backward scattered radiation, such as by an annual detector, and/or providing feedback control to the means for directing radiation.

Such is a well appreciated feature, as almost in the same time frame a user is capable of checking results of the sculpting, and/or optionally adjusting said sculpting, if considered necessary. The sculpting can be followed “real time”, effectively after removal of, e.g., each atom. It is noted that only a small delay is involved, e.g., a time needed to form an image, to process data, and the like. The delay is therefore in an order of μsec-msec. The feedback control loop may comprise software for analyzing an image obtained, e.g. in view of quality of the sculpture, in view of a crystallographic direction to be followed during sculpting, etc. The feedback loop and/or computer associated therewith may further comprise a pre-determined shape to be sculpted, which shape is than sculpted according to the present method. The feedback may also provide valuable information, e.g. on quality, of an intermediate product being formed. Such is not available in the prior art.

The image formed accordingly may also be used as a means for quality control. In an example a formed imaged may be characterized e.g. in terms of position, orientation, shape, size, width, length, etc. Using an electron microscope this can be done with high precision, e.g. with an accuracy of about 0.01 nm. Also one or more images may be formed during sculpting, and/or in between sculpting, and/or in a final stage.

The present invention relates in a second aspect to a free-standing layer comprising one or more nanostructures formed therein obtainable by the present method, wherein the one or more nanostructures are defined with a precision of less than 1 nm, preferably less than 0.5 nm, more preferably less than 0.25 nm, such as of about 0.1 nm, wherein the one or more nanostructures are selected from the group comprising a hole, a bridge, two or more parallel bridges, a ribbon, a bridge in a crystallographic direction [hkl], and combinations thereof, and wherein the layer is from one monolayer—10 monolayers thick, preferably from 1-5 monolayers, such as from 1-2 monolayers.

In an exemplary embodiment of the present layer the layer is a monolayer of graphene, a bilayer of graphene, or a layer of graphene on a layer of a further material, such as BN. As such combinations of one or more layers having the same, similar, or different materials.

The layer may for instance comprise one or more of nanoholes, nanoslits, e.g., along a crystallographic direction [hkl], nanobridges, e.g. between a first and a second part of the layer, and nano rasters, such as a hexagonal or trigonal raster comprising one or more holes therein.

The layer is preferably one atom or molecule thick, optional two atoms or molecules. A somewhat thicker layer provides e.g., better mechanical strength. A monolayer has somewhat better electro-magnetically properties.

The layer may also relate to a so-called 2-dimensional crystal or the like. In other words, a crystallographic layer may be formed according to the invention, wherein an option of self-repair is available.

The present invention relates in a third aspect to a sensor for detecting charged species in a fluid, comprising a free-standing layer according to the invention.

In an exemplary embodiment the sensor comprises an electro-magnetically conducting layer.

In an exemplary embodiment the present sensor further comprises an electrical power supply, and a means for detecting direct or indirect fluctuations in one or more of electrical field and magnetic field, such as in current, resistance, potential, charge, inductance, capacitance, magnetic field, frequency, power and flux. As fluctuations are typically very small, the sensitivity and selectivity of the means for detecting electro-magnetic variations is preferably very high. In principle such means and attributes for measuring e.g., nano amperes or lower are at present available.

The layer of the sensor may comprise one or more nanostructures. Typically the layer is somewhat less wide in a middle thereof. The support beneath said middle typically will comprise a hole for letting e.g. a fluid pass through. The hole in the support is typically at least one order of magnitude larger than the nanostructures.

In an example the middle part of the layer may comprise one or more bridges, the one or more bridges preferably being aligned. A fluid may pass alongside said bridges, optionally causing a variation in electro-magnetic behavior thereof. The variation can be measured and is indicative for the nature of the fluid, and/or species therein, passing by. The layer may be provided with a thin conductor attached thereto, such as a metal wire, such as a (nm) Pt wire. Such provides improved reliability and reproducibility.

In an exemplary embodiment the present sensor is for detecting one or more of a single ion, a DNA-base pair, a RNA-base pair, an enzyme, a protein, a nucleotide, a gene, a molecule, such as ethene, CO₂, CO, poisonous gas, O₂, and volatiles, a plasmid, and a virus. With the present sensor and/or method, and typically a calibration curve or the like, the above ions and molecules can be analyzed.

The present invention relates in a fourth aspect to a use of a sensor according to the invention for detecting one or more of a single ion, a DNA-base pair, a RNA-base pair, an enzyme, a protein, a nucleotide, a gene, a molecule, a plasmid, and a virus.

The present invention relates in a fifth aspect to a method of detecting a species such as one or more of single ion, a DNA-base pair, a RNA-base pair, an enzyme, a protein, a nucleotide, a gene, a molecule, a plasmid, and a virus, comprising the steps of: providing a sensor according to the invention, providing a sample comprising the species, detecting presence of the species, and optionally one or more further characteristics of the species, such as concentration, base-pair sequence, or absence of the species.

Thereby the present invention provides a solution to one or more of the above mentioned problems.

Advantages of the present description are detailed throughout the description.

### EXAMPLES

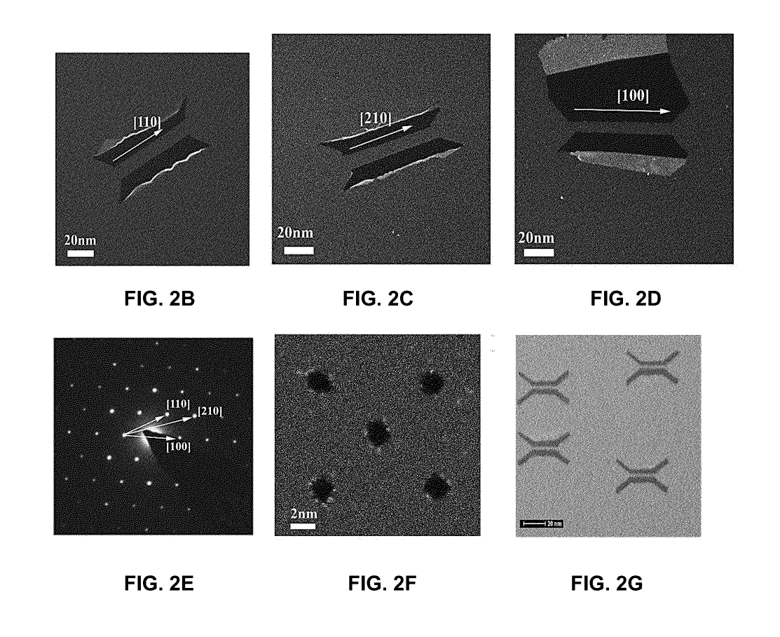

The present inventors operated a transmission electron microscope in Scanning Transmission Electron Microscopy mode at 300 keV and 200 keV, in which electrons are focused into a fine spot of 0.1 nm. In this mode, the electron dose exposed onto graphene (4) could be simply controlled over a time the electron probe residual in a given position, the dwell time. Thus, setting different scanning dwell times, the present inventors achieved a slow scan for a destructive sculpting and a fast scan for a non-destructive imaging of the sculpted structure without a need for changing electron beam condition (300 keV beam energy and 0.15 nA beam current in most of the experiment). Optionally sculpting is chemically assisted. A schematic diagram is given in FIG. 2a, showing a present configuration of feed-control sculpting in STEM mode, comprising scanning coils (1), incident electrons (2), back scattered electrons (3), heating coils (5), preferably made of Pt, a SiN support (6), an annual detector (8), an image forming step (9) typically using a computer, and a feedback control (10). The image is formed (9) by fast scanning a sub-Angstrom electron probe over an interested region and collecting all the forward scattered electrons (7) using an annular detector (8). The dwell time of imaging is usually set as 5˜30 μs, giving a radiation dose as ˜10⁻⁴ electrons/atom. It has been found that the possibility of inducing one carbon atom displacement by a 300 keV electron is less than 10⁻⁷, and therefore the total sputtering possibility by the radiation dose during imaging process is rather rare (less than 10⁻³). It has been found experimentally that easy achieving of non-destructive imaging is preferred for fast and controllable sculpting, because one can immediately image the geometries of a sculpted pattern without inducing extra unwanted electron beam damage. Therefore, based on imaging a feedback (10) control is built to correctly and precisely adjust the sculpting process. The typical sculpted nano-structures of graphene (4) are respectively shown in FIG. 2b-f, including three nano-ribbons with defined ribbon directions along crystallographic [100], [110] and [210] directions giving edges of a zigzag, armchair and a mixed type pattern, respectively, and an ordered nano-hole pattern, with each nano-hole of the same diameter (2 nm). The width of the nano-ribbon and the diameter of the nano-holes can be controlled within sub-nano-meter accuracy and they can be easily reproduced.

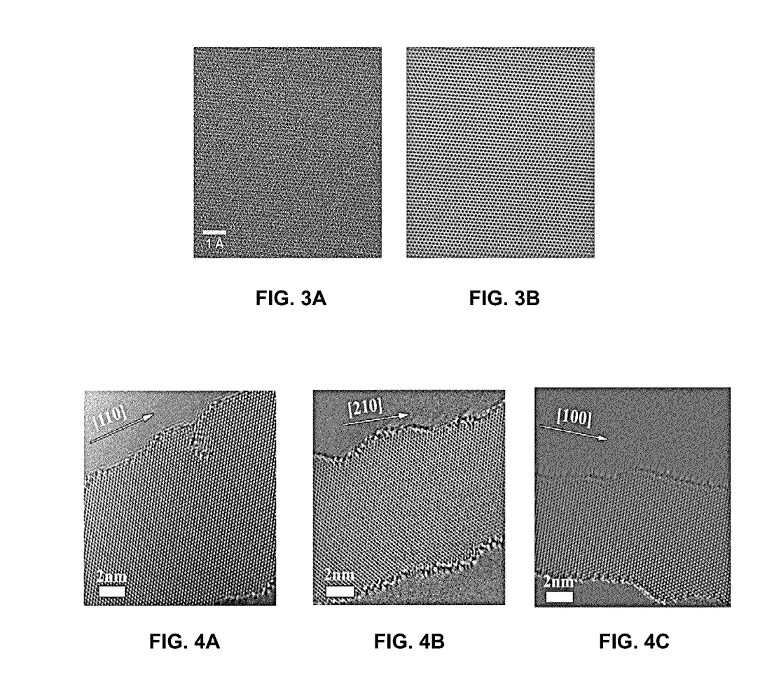

Another important component of controllable sculpting is that the sample is heated above, e.g., 500° C., because this allows a self-repairing effect. During a fast scan for imaging, the chance of inducing e.g. carbon knock out damage is found to be rare, but it may still initialize few point defects of carbon vacancies on graphene (the density of carbon vacancies is typically around 10⁻³). It has been found that without heating the sample, around these point defects e-beam damage can be easily developed in a next scan. However, at a high temperature protecting full integrity of a graphene lattice in an imaging process is protected. This effect of heating can be visualized from FIG. 3, in which an atomic resolution STEM imaging at 300 kV of defect free graphene lattice using a rather long dwell time of 240 μs.

Whereas a fast scan of STEM provides an easy imaging of a shape of sculpted structures, atomic resolution imaging of what was made, for instance, crystallinity and sharpness of an edge is more difficult to obtain, because of a long exposure time needed; for instance, a 300 keV electron beam certainly changes edges of a sculpted structure (SI). For this reason, it has been established to image details using an acceleration voltage of less than ˜100 keV, which is found to be about the graphene damage threshold.

FIG. 4 shows HREM images of sculpted ribbons at 80 kV and a graphene sample is also heated to 600° C. for further protecting the edge of a ribbon. It is clearly observed that crystallinity of the graphene lattice stops close to the nano-ribbon edge. Inventors have often observed that an atomic sharp edge was obtainable for a [100] direction (zigzag) and [110] direction (armchair). However, despite of sculpting with the same setting, the edges of the ribbons along other orientations are not atomically sharp. An instability of the edge along these directions is observed. It is therefore believed that a stable edge in a random direction can only be constructed by combining two stable zigzag and armchair edges, resulted in the roughness of the edge.

In summary, inventors have demonstrated a full control of the scanning electron beam technique to sculpt mono-layer graphene into size, site (position) and orientation specific nano-patterns, an advance in view of the prior art that allows automatic pattern writing on graphene sheet and the like, e.g., for large scale application. This capability opens new applications of graphene in nano-electronics and nanophysics.

### Sample Preparation and Transferring:

Graphene flakes were prepared by exfoliation of natural graphite (NGS graphite) on a 285 nm thermally grown SiO₂Si wafer.

Graphene flakes of interest were selected using optical interference microscopy. A selected graphene flake was then transferred on top of a hole in a supporting SiN membrane using a wedging transfer technique. The crystallinity and the single-layer graphene were further checked using electron diffraction.

A heating holder with a MEMS heater was used for in-situ experiments.

For in-situ heating, a SiN membrane was used with an embedded, coiled Pt wire. In the SiN membrane, a 2 μm diameter hole was made with a focused ion beam through the Pt wire to allow substrate-free TEM imaging of the graphene.

It is noted that it has been found experimentally that the very low heat capacity of the heater results in very low thermal drift, which enables stabile scanning microscopy electron microscopy imaging at elevated temperature.

### Parameters for STEM Sculpting and Imaging:

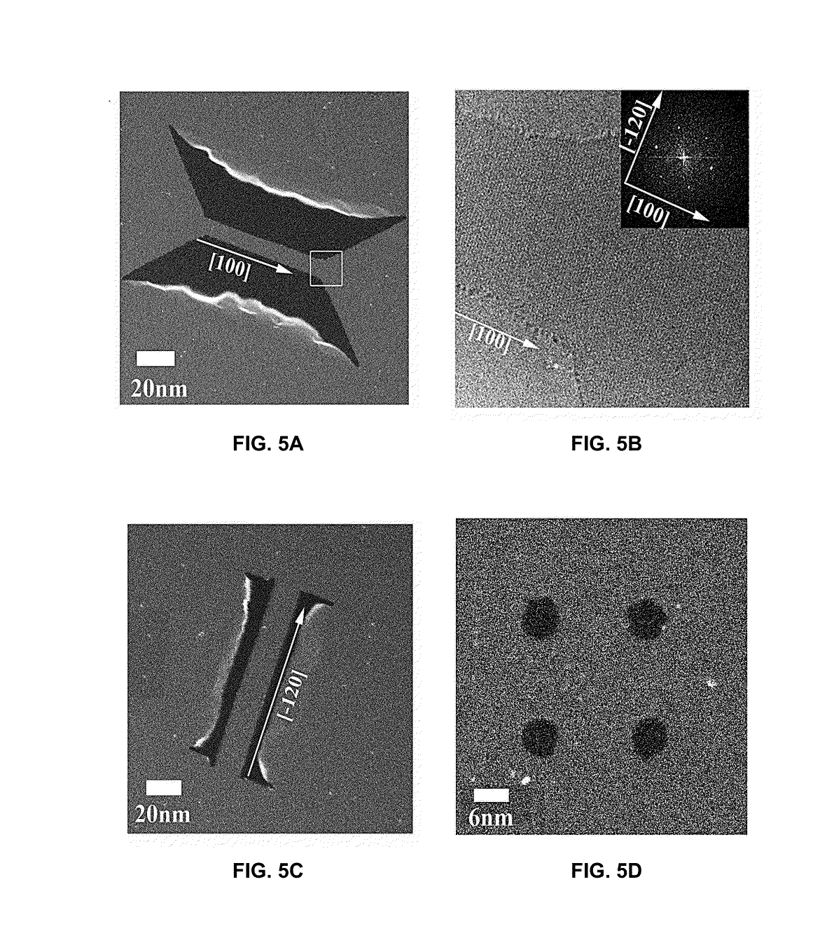

STEM imaging of nano-patterns (in FIG. 5) was performed in a cubed FEI Titan microscope with a post-specimen was corrector operated at 300 keV. Spherical aberration is always set below 1 micron (μm). A convergent angle of focused electron is set at 10 mrad for achieving a very fine electron beam. The camera length is set to 470 mm in order to allow the annual detector to record a maximum number of diffracted electron beams of graphene, in order to obtain a good signal. The electron beam current was set at ˜0.15 nA for both STEM imaging and sculpting. The time was set at 5˜30 μs for imaging and 10 ms for sculpting.

A HRSTEM image of monolayer graphene was obtained in a Titan3 G2 60-300 TEM, equipped with both image and probe correctors and a monochrornator. The microscope was operated at 300 kV with a beam current at 0.2 nA. The convergent angle is set at 20 mrad. For collecting a maximum number of diffracted electron beams of graphene best signal, the camera length is set at 185 mm. The imaging time was set at 240 μs, resulting in a total of 52 seconds for recording a 512*512 pixels image.

The high resolution transmission electron microscopy (HRTEM) was performed in a Titan 60-300 PICO TEM equipped with a high brightness electron gun, Cs probe correctors and a monochromator. unit together with a Cs-Cc achro-aplanat image corrector. The microscope was operated at 80 kV. No apparent beam damage was observed during image recording, however, a longer expose time of nano-ribbons under a high energy high current electron beam may cause breakage of ribbons. Inventors took 10 images for each nano-ribbon with 2 seconds exposure time using a 4 k by 4 k Gatan CCD camera with a binning set to 2. Then these image sequences are aligned and summed up to give an image with a high signal-noise ratio, such as by using software (e.g., ImageJ).

### Control of Knock Out Damage:

While operating in STEM mode, an easy control over a time an electron probe resides in a given position, the dwell time, was achieved. By tuning the dwell time, a control of a total number of electrons exposed on a carbon atom (dose) by giving a fixed electron beam current (typically 0.1-0.2 nA) was obtained. It is noted that only a very tiny portion of incident high-energy electrons exactly hit a core of an (C) atom and are then back-scattered, The electrons can induce a so-called knock out damage. Most of incident electrons are only slightly scattered and can be used to form an STEM image. The huge ratio between forward forward-scattered electrons for imaging and the back-scattered electrons for sculpting allows inventors to set a dwell time to less than a critical value. Under a critical dwell time the fraction of the weakly scattered electrons is found to be sufficient to provide a good contrast of an STEM image, whereas it has been found that the fraction of the back scattered electrons hardly create carbon vacancies. Such vacancies if required could be self-repaired when the graphene is at elevated temperatures.

### Control Parameters for STEM Sculpting and Imaging:

When an electron beam is fixed at one position (no scanning but static), it is believed that electron beam damage relies on how long the electron beam stays on said position, which is may be the dwell time only. When the dwell time is longer than a critical time, it has been found that an electron beam will create a hole around the electron beam position. The size of hole grows with increasing dwell time, up to a final size, determined by a whole by electrons exposed region, usually being about several nanometers (in diameter) in an STEM mode. It is noted that a real electron beam exposed region is typically much larger than a “spot”, where only 80% of a number of electrons are focused at. It is observed that once vacancies initially are created at a center spot, carbon atoms near the vacancies are not fully bonded and can therefore be removed easier. Thus an by an e-beam created hole can grow out to the whole by electrons exposed area, even though an outer region thereof is only weakly exposed by electrons. For this reason, an electron beam is preferably blocked or kept in fast scanning mode for protecting integrity of, e.g., graphene, except when sculpting is required.

It has been found that when an electron beam is scanning on e.g. graphene, another parameter, namely scanning resolution, will also play an important role in e-beam damage. During scanning, an electron beam does not continuously move over a sample. Instead, the electron beam stays at a given position for a certain time, jumps to a next position, being a certain distance away from the previous position. This step size between neighboring scanned positions is typically termed as the scanning resolution. In order to have a continuous cutting through of graphene lattice, it has been found that the scanning resolution cannot be set too large, e.g., not larger than a size of a hole etched by an e-beam. Otherwise only discrete holes will be created. On the other hand, it has been found that a small scanning step size results in overlapping of a by electrons exposed region for neighboring scanning points. The common area of these points suffers in the example double electron exposure or even more. This is similar to an effect of increasing dwell time. Thus, in the sculpting process, a smaller scanning resolution will result in a wider cutting line, which is not desired. More seriously in the imaging process, if a very small scanning step size is used, heavy overlapping is thereby induced, which may dramatically increase an effective dwell time, resulting unexpected e-beam damage in imaging process. This often happens for recording an atomic resolution STEM. In order to reach a high magnification for imaging carbon atom, the scanning resolution is normally set as 0.15 Å/pixel. Since the size of one carbon atom is 1.4 Å (approximated by using C—C bonding length), n effective electron exposed dwell time for one carbon atom will in an example be 10 times higher than a static dwell time setting. In the present HRSTEM experiment, a relatively long dwell time of 240 μs was once used for achieving single C atom contrast, This resulted in actually exposing the single one carbon atom by an electron beam up to ˜2.4 ms, which is comparable to the time used for sculpting (10 ms). Thus, although inventors had observed that graphene sheet keeps its integrity after taking 3-4 HRSTEM images from one area of the graphene, it has been observed that further scanning of same area always produces collapse of graphene lattice, being undesirable.

The invention is further detailed by the accompanying figures, which are exemplary and explanatory of nature and are not limiting the scope of the invention. To the person skilled in the art it may be clear that many variants, being obvious or not, may be conceivable falling within the scope of protection, defined by the present claims.

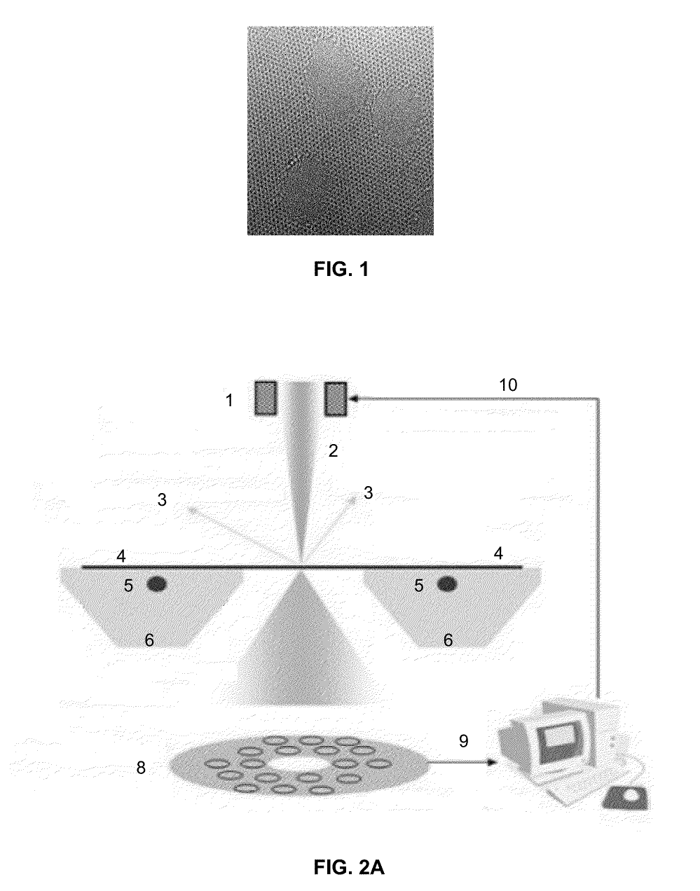

FIG. 1 shows a STEM of monolayer graphene.

FIG. 2 Schematic diagram sketches the configuration of sculpting graphene using scanning transmission electron microscopy: a high energy electron beam is focused and scanned on a graphene sheet. Back scattered electrons induce a knockout of carbon atoms, used for sculpting the nano-pattern and forward scattered electrons are collected to form a STEM image, which may be used for control of the sculpting process. The graphene sheet is laid on a SiN MEMS, which is heated by embedded Pt coils. In this configuration, nano-ribbons are created along three specific orientations, [100] (B) [210] (C) and [110] (D), referenced to the diffraction of the graphene (E); An ordered nano-hole pattern with controlled diameter size of about 2 nm is also obtained (F); further a bridge like structure of about 20 nm are shown, which structure can be produced with high reproducibility and accuracy (G).

FIG. 3 shows a high resolution scanning transmission electron microscopy of a mono-layer graphene heated at 650° C. being recorded by operating the microscope at 300 kV (A). The denoised image (B), being an image processed from image (A), clearly indicates a nice arrangement of carbon hexagon rings without visible atoms vacancies. Such is highly desired.

FIG. 4 shows a high resolution electron microscopy of Nano-ribbons obtained at 80 kV.

### Reproducibility of Controllable Sculpting:

The inventors have performed controllable sculpting on different graphene samples. A good repeatability and accuracy is achieved. Another example is given in FIG. 5. A further way of making a nano-ribbon is demonstrated.

FIG. 5 (A) shows a STEM image for an electron etched nano-ribbon with defined ribbon orientation along [100]. Figure (B) shows a HREM image of a part of the ribbon of figure (A) for indicating crystallinity of the ribbon edge and an inset of FFT (Fast Fourier Transform) of the image at right upper corner is provided showing the crystal orientation of the graphene. The illumination region of (B) is also outlined by a white enclosed frame in (A). Figure (C) shows a STEM image of another ribbon along [-120]. Figure (D) shows an ordered pattern of nano-holes with 6 nm diameter.

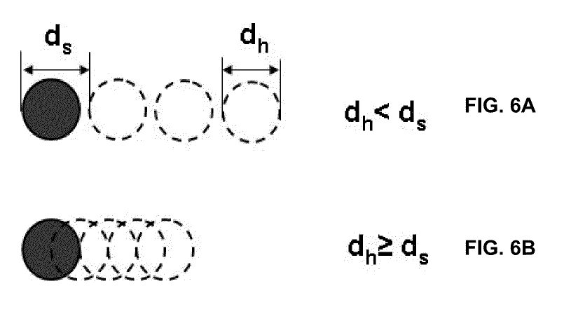

FIG. 6 shows the influence of scanning resolution. ds denotes the scanning resolution that a distance between two neighboring electron beams exposed positions in the scanning. dh denotes a size of an e-beam etched hole on a graphene sheet. In order to get a continuous cutting through graphene, the scanning resolution is set to be not less than a certain value. In FIG. 6a dh<ds, implying that the scanning resolution is larger than the size of a hole. A first hole is sculpted, followed by subsequent holes 2-4. As such, during sculpting, some material may remain in between holes. On the other hand, in FIG. 6b, dh≧ds, implying that the scanning resolution is smaller than the size of a hole, Therewith a “continuous” removal of material is obtained (holes 1-5).

The invention although described in detailed explanatory context may be best understood in conjunction with the accompanying figures.

What is claimed is:

1. A method for removing a high definition nanostructure in a partly free-standing layer with a thickness of less than 5 nm, comprising the steps of:
a) providing a radiation source, a means for high precision directing radiation, a sample, the sample comprising the free-standing layer, a support for largely supporting the layer, and one or more means for self-repairing of the layer,
b) activating said means for self-repairing, and
c) focusing said radiation in a bundle on the sample during a period sufficient for removing the high definition nanostructure.

2. The method according to claim 1, wherein the radiation source is an electron gun of an electron microscope.

3. The method according to claim 1, wherein radiation is focused to an area of less than 2 nm.

4. The method according to claim 1, wherein an energy used for removing one atom in the layer is from 1*10⁻¹⁸ J-1*10⁻¹⁶ J.

5. The method according to claim 1, wherein sculpting per single point is performed during a period of 0.01-1000 mseconds.

6. The method according to claim 1, wherein after focusing:
d) the radiation bundle is moved to a next position on the layer.

7. The method according to claim 6, wherein the bundle is moved from a first to a further position, which movement is repeated from 1-10*10⁹ times.

8. The method according to claim 1, wherein further an image is formed of the layer.

9. A free-standing layer comprising one or more nanostructures formed therein obtainable by a method according to claim 1, wherein:
the one or more nanostructures are defined with a precision of less than 1 nm,
the one or more nanostructures are selected from the group consisting of a hole, a bridge, two or more parallel bridges, a ribbon, a bridge in a crystallographic direction [hkl], and combinations thereof, and
the layer is from one monolayer—10 mono-layers thick.

10. The free-standing layer according to claim 9, wherein the layer is a monolayer of graphene, a bilayer of graphene, or a layer of graphene on a layer of a further material.

11. A sensor for detecting species in a fluid, comprising a free-standing layer according to claim 9.

12. The sensor according to claim 11, further comprising an electrical power supply and a means for detecting direct or indirect fluctuations in one or more of electrical field and magnetic field.

13. The sensor according to claim 11 for detecting one or more of a single ion, a DNA-base pair, a RNA-base pair, an enzyme, a protein, a nucleotide, a gene, a molecule, a plasmid, and a virus.

14. Use of a sensor according to claim 11 for detecting one or more of a single ion, a DNA-base pair, a RNA-base pair, an enzyme, a protein, a nucleotide, a gene, a molecule, a plasmid, and a virus.

15. A method of detecting a species such as one or more of a single ion, a DNA-base pair, a RNA-base pair, an enzyme, a protein, a nucleotide, a gene, a molecule, a plasmid, and a virus, comprising the steps of:
providing a sensor according to claim 11,
providing a sample comprising the species, and
detecting presence of the species.

16. The method according to claim 15, additionally comprising detecting one or more further characteristics of the species selected from the group consisting of concentration, base-pair sequence, and absence of the species.

17. The method according to claim 1, wherein the layer is a monolayer.

18. The method according to claim 1, wherein the layer comprises graphene.

19. The method according to claim 1, wherein the means for self-repairing comprises heating means.

20. The method according to claim 19, wherein the heating means increases temperature of the layer to above 400° C.
